(19) United States
(12) Patent Application Publication
(10) Pub. No.: US 2026/0207711 A1
(43) Pub. Date: Jul. 23, 2026

(54) FORMULATIONS, DEVICES AND METHODS FOR A GLP-1 AGONIST

(71) Applicant: Nano Precision Medical, Inc., Alameda, CA (US)

(72) Inventors: Wouter Roorda, Alameda, CA (US); Adam Mendelsohn, Alameda, CA (US); Lien Vonguyen, Alameda, CA (US)

(73) Assignee: Nano Precision Medical, Inc., Alameda, CA (US)

(21) Appl. No.: 19/140,705

(22) PCT Filed: Jan. 4, 2024
(86) PCT No.: PCT/US2024/010303

§ 371 Date: Jun. 18, 2025

Related U.S. Application Data
(60) Provisional application No. 63/437,541, filed on Jan. 6, 2023.
(60) Provisional application No. 63/523,458, filed on Jun. 27, 2023.

(51) Int. Cl. A61K 38/26; A61K 9/00; A61K 47/40

(52) U.S. Cl. CPC A61K 38/26; A61K 9/0024; A61K 47/40

(57) ABSTRACT

The present disclosure provides formulations, methods and implantable devices that improve patient compliance by releasing a desired therapeutic agent such as semaglutide for a long time period, such as 1 year or more. The semaglutide concentration (w/w) is more than 1%, such as about 1.1% w/w to about 35% w/w.

### CROSS-REFERENCES TO RELATED APPLICATIONS

This application claims priority to US Provisional Patent Application Nos: 63/437,541, filed Jan. 6, 2023, and 63/523,458, filed Jun. 27, 2023, the teachings of which are hereby incorporated by reference in their entireties for all purposes.

### BACKGROUND

Many subjects, humans as well as animals need long-term treatment with therapeutic agents. In order to improve adherence or compliance, many subjects would benefit from the adherence provided by an implantable device releasing a desired therapeutic agent at a desired rate for an extended period of time. Despite many years of research there is still a need for the development of such devices, and specifically for methods to control the rate of release of therapeutic agents from such devices upon implantation in a subject to be treated.

In view of the foregoing, the present disclosure provides formulations, methods and implantable devices that improve patient compliance by releasing a desired therapeutic agent for a long period of time, even 1 year or more as well as offering other advantages as well.

### BRIEF SUMMARY

The present disclosure provides implantable drug delivery devices and pertains to the long-term treatment of subjects with implantable devices providing a sustained delivery of one or more therapeutic agents such as an incretin mimetic at a controlled rate. The compositions, implantable drug delivery systems and methods of using the therapeutic agents are useful for treating a disease, such as diabetes and for administering to a subject in need of treatment by the therapeutic agent.

In certain aspects, embodiments comprise a formulation having a solvent, the formulation comprising semaglutide having a concentration, wherein the semaglutide concentration (w/w) is more than about 1%, such as 1.1% to about 35% w/w or more.

In certain aspects, embodiments comprise a formulation wherein the semaglutide concentration (w/w) is one of at least 2%, at least 5%, at least 10%, at least 15%, at least 20%, at least 25% and at least 30% (w/w).

In certain aspects, embodiments comprise a formulation of semaglutide wherein the pH of the formulation is between 6 and 10.

In certain aspects, embodiments comprise a formulation of semaglutide wherein substantially no additional pharmaceutical excipients are present.

In certain aspects, embodiments comprise a formulation of semaglutide wherein a tonicity agent is present.

In certain aspects, embodiments comprise a formulation of semaglutide wherein the tonicity agent is a tonicity agent other than sodium chloride. The concentration of the tonicity agent can be from about 0.1% to about 10% w/w.

In certain aspects, embodiments comprise a formulation of semaglutide wherein the tonicity agent is potassium chloride.

In certain aspects, embodiments comprise a formulation of semaglutide wherein the solvent is water.

In certain aspects, embodiments comprise a formulation of semaglutide wherein the solvent is an organic solvent or a water-organic solvent mixture.

In certain aspects, embodiments comprise a formulation of semaglutide wherein an additional pharmaceutical excipient is present.

In certain aspects, embodiments comprise a formulation of semaglutide wherein the additional pharmaceutical excipient is one or more of an antimicrobial, a surfactant, an antioxidant, a free radical scavenger and a peptide stabilizing agent.

In certain aspects, embodiments comprise a formulation of semaglutide wherein the peptide stabilizing agent is one or more selected from the group of a cyclodextrin and/or an amino acid.

In certain aspects, embodiments comprise a formulation of semaglutide wherein the cyclodextrin is hydroxy-propyl alpha cyclodextrin.

In certain aspects, embodiments comprise a formulation of semaglutide wherein the amino acid is histidine.

In certain aspects, embodiments comprise a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the semaglutide concentration (w/w) is more than about 1%.

In certain aspects, embodiments comprise a device for sustained release of semaglutide wherein the semaglutide concentration (w/w) is one of at least 2%, at least 5%, at least 10%, at least 15%, at least 20%, at least 25% and at least 30%.

In certain aspects, embodiments comprise a device for sustained release of semaglutide wherein the pH is between about 6 and about 10.

In certain aspects, embodiments comprise a device for sustained release of semaglutide wherein substantially no additional pharmaceutical excipients are present.

In certain aspects, embodiments comprise a device for sustained release of semaglutide wherein a tonicity agent is present.

In certain aspects, embodiments comprise a device for sustained release of semaglutide wherein the tonicity agent is a tonicity agent other than sodium chloride.

In certain aspects, embodiments comprise a device for sustained release of semaglutide wherein the tonicity agent is potassium chloride.

In certain aspects, embodiments comprise a device for sustained release of semaglutide wherein the solvent is water.

In certain aspects, embodiments comprise a device for sustained release of semaglutide wherein the solvent is an organic solvent or a water-organic solvent mixture.

In certain aspects, embodiments comprise a device for sustained release of semaglutide wherein n additional pharmaceutical excipient is present.

In certain aspects, embodiments comprise a device for sustained release of semaglutide wherein the additional pharmaceutical excipient is one or more selected from the group of an antimicrobial, a surfactant, an antioxidant, a free radical scavenger and a peptide stabilizing agent.

In certain aspects, embodiments comprise a device for sustained release of semaglutide wherein the peptide stabilizing agent is one or more selected from the of the group of a cyclodextrin and an amino acid.

In certain aspects, embodiments comprise a device for sustained release of semaglutide wherein the cyclodextrin is hydroxy-propyl alpha cyclodextrin.

In certain aspects, embodiments comprise a device for sustained release of semaglutide wherein the amino acid is histidine.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the semaglutide concentration (w/w) is more than 1%.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the semaglutide concentration (w/w) is one of at least 2%, at least 5%, at least 10%, at least 15%, at least 20%, at least 25% and at least 30%.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir, wherein the pH is between about 6 and about 10.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein substantially no additional pharmaceutical excipients are present.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein a tonicity agent is present.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the tonicity agent is a tonicity agent other than sodium chloride.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the tonicity agent is potassium chloride.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the solvent is water.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the solvent is an organic solvent or a water-organic solvent mixture.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein an additional pharmaceutical excipient is present.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the additional pharmaceutical excipient is one or more of an antimicrobial, a surfactant, an anti-oxidant, a free radical scavenger and a peptide stabilizing agent.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the peptide stabilizing agent is one or more of the group of a cyclodextrin and an amino acid.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the cyclodextrin is hydroxy-propyl alpha cyclodextrin.

In certain aspects, embodiments comprise a method for treating a subject with semaglutide, comprising providing a device for sustained release of semaglutide, the device comprising a capsule configured for implantation and having a reservoir; a nanoporous membrane with a plurality of pores; a liquid formulation of semaglutide having a solvent and having a concentration of semaglutide disposed within the reservoir; wherein the amino acid is histidine.

In In certain aspects, embodiments of the disclosure, excipients in the formulation modulate the release rate of the pharmaceutical agent. Modulation of release rate by surfactants has been disclosed in PCT/US2023/023845, filed May 30, 2023, which is incorporated herein by reference. It has now been found that other excipients may modulate release rate as well. For instance, the presence of hydroxy-propyl a cyclodextrin was found to flatten the release rate profile of semaglutide. Generally, flattening a long-term release rate profile of therapeutic agents is considered desirable.

Cyclodextrins are beaker-shaped oligosaccharides, with a hydrophobic interior and a hydrophilic exterior. They are typically used to encapsulate small hydrophobic molecules, much smaller than GLP-1 analogs, to improve their water-solubility. The most common cyclodextrins are α, β, and γ cyclodextrin. To improve their own water-solubility, they are often derivatized with a hydroxypropyl group.

These and other aspects, objects and advantages will become more apparent when read with the figures and detailed description which follow.

### BRIEF DESCRIPTION OF THE DRAWINGS

FIG. 1 represents a device of the disclosure.

FIG. 2 represents a release rate profile of a first embodiment of the disclosure.

FIG. 3 represents a release rate profile of a second embodiment of the disclosure.

FIG. 4 represents a release rate profile of a third embodiment of the disclosure.

FIG. 5 represents a release rate profile of an embodiment of the disclosure.

### DETAILED DESCRIPTION

### Definitions

The terms “polypeptides” refers to molecules with a backbone chain of 2 or more amino acid residues. Some polypeptides may have additional associated groups, such as metal ions in metalloproteins, small organic molecules such as in heme proteins, or carbohydrate groups such as in glycoproteins.

The terms “peptides” and “proteins” refers to subgroups of polypeptides. In this disclosure the definition of peptides and proteins follows the practice of the United States Food and Drug Administration, the FDA, which defines peptides as polypeptides with up to 40 amino acid residues, and proteins as polypeptides with more than 40 amino acid residues.

The term “incretin mimetics” refers to agents that act like incretin hormones such as glucagon-like peptide-1 (GLP-1). They bind to GLP-1 receptors and stimulate glucose dependent insulin release, therefore acting as antihyperglycemics. Incretin mimetics also suppress appetite and inhibit glucagon secretion. They slow gastric emptying and as a result prevent a steep rise in post-prandial blood glucose levels. In addition, incretin mimetics also include compounds that possess GLP-1 agonism as well as other complimentary antagonism or agonism properties, including, but not limited to, GLP and/or Glucagon antagonisms.

The term “formulation of a therapeutic agent” refers to the actual state in which a therapeutic agent is present in a product or in a product fabrication intermediate, and includes the therapeutic agent, plus, optionally, any used additional therapeutic agents, any used formulation excipients and any used formulation solvents.

The term “membrane” refers to a permeable structure allowing mass transport of molecules from one side of the structure to the other through the structure.

The term “porous membranes” refers to membranes characterized by the presence of a two-phase system, in which membrane matrix material represents one phase, typically a continuous phase, which is permeated by open channels extending from one side of the membrane to the other, and filled with a second phase, often a fluid phase, through which mass transport through the membrane can take place.

The terms “dense” or “non-porous membranes” refers to membranes without fluid filled pores. In such membranes mass transport may take place by a dissolution-diffusion mechanism, in which therapeutic agents permeate the membrane by dissolving in the membrane material itself, and diffusing through it.

The terms “nanoporous membrane” and “nanopore membrane” are used interchangeably, and refer to porous membranes in which the pores have a smallest diameter of less than 1000 nanometer.

The term “nanotube membrane” refers to a nanoporous membrane, wherein pores are formed by an array of nanotubes.

The term “titania nanotube membrane” refers to an array of titania nanotubes on a titanium substrate where at least a portion of the titania nanotubes are open at both ends and capable of allowing diffusion from one side of the membrane to the other through the titania nanotubes. In certain instances, the titania nanotube membrane has two faces or sides. A first face or side having an array of titania nanotubes and a second face or side of a titanium substrate. In certain aspects, the array of titania nanotubes is grown on the titanium substrate by electrochemical anodization.

The term “molecular diameter” of a polymer refers to the diameter of the sphere of gyration of the polymer, which is a physical measure of the size of a molecule and is defined as two times the mass weighted average distance from the core of a molecule to each mass element in the molecule.

The terms “Stokes diameter” or “hydrodynamic diameter” refers to the dimension of a molecule plus its associated water molecules as it moves through an aqueous solution and is defined as the radius of an equivalent hard sphere diffusing at the same rate as the molecule under observation.

The terms “fluid” and “fluid form” as used in this disclosure refers to flowable states of matter and includes, but is not limited to gases, solutions, suspensions, emulsions, colloids, dispersions and the like.

The term “fluid contact” refers to an entity being in contact with a fluid.

The term “neutral pH” refers to a pH between 6.5 and 7.5 such as 7.

### EMBODIMENTS

Embodiments of the disclosure include devices, formulations and methods to control the rate of release of one or more therapeutic agents from such devices. Additionally, embodiments of the disclosure include methods of treatment of a subject with devices and formulations of the disclosure. US Patent Application Publication Nos. 20210246271 and 20220008345 are incorporated into this disclosure in their entirety by reference.

### Devices

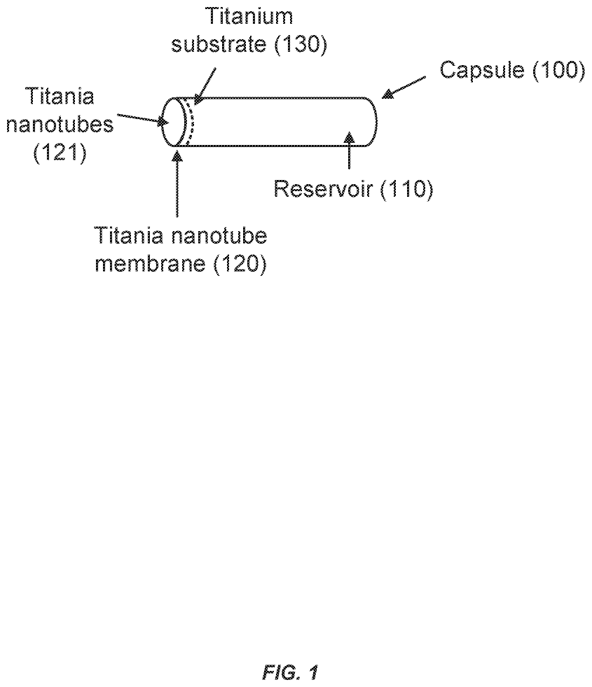

As illustrated in FIG. 1, devices of the disclosure include a capsule 100 suitable for implantation, wherein the capsule has a reservoir 110 suitable for holding a therapeutic agent and, optionally, a pH controlling agent. In certain embodiments more than one reservoir is present. The capsule may be made of any suitable biocompatible material. In certain embodiments the capsule is made of a medical grade metal, such as titanium or stainless steel, or of a medical grade polymeric material, such as silicone, polyurethane, polyacrylate, polyolefin, polyester, polyamide and the like. In embodiments the capsule is made of multiple materials. In some embodiments of the disclosure, the capsule is made of titanium.

Devices of the disclosure have at least one membrane 120, as described herein, attached to the capsule and in fluid contact with the reservoir, wherein the membrane provides a pathway for mass transport of a therapeutic agent included within the reservoir out of that reservoir and into the body of a subject into which the capsule has been implanted. In this disclosure “attached to the capsule” refers to a component being fixed in place with respect to the capsule, and connected to the capsule directly or indirectly, by using any suitable means, including by welding, gluing, press-fitting and by using threaded means, or by any combination of these. In the case of membranes as described in U.S. Pat. No. 9,814,867, and as illustrated in FIG. 1, the nanotube membranes are part of an array of nanotubes 121, some of which are still attached to the titanium substrate 130 from which they were grown, and the substrate may be attached to the capsule. At least some of the nanotubes are open on both sides, to allow for mass transport of a therapeutic agent out of the reservoir and when implanted, out of the reservoir and into a subject. In one aspect, the membrane 120 is a titania nanotube membrane 120, which has two faces or sides. A first face or side having an array of titania nanotubes 121 and a second face or side of a titanium substrate 130. FIG. 1 shows the membrane attached to the capsule with the titanium substrate 130 facing towards the reservoir of the device. The device is configured to be implanted in a subject.

### Membranes

Embodiments of the disclosure include at least one membrane providing a pathway for mass transport of a therapeutic agent out of a reservoir of a device of the disclosure.

A wide variety of membranes can be used in embodiments of the present disclosure. Membranes of the disclosure include dense and porous membranes; porous membranes include nanoporous membranes and nanotube membranes.

Suitable materials for membranes of the disclosure include organic and inorganic materials, polymers, ceramics, metals, metal oxides and combinations thereof. Suitable materials for the membrane include silicon, silica, titanium and titania. In some embodiments, the membrane is a nanoporous membrane. In some embodiments the membrane is a nanotube membrane. In some embodiments the membrane is a titania nanotube membrane.

Embodiments of the disclosure are particularly useful as sustained delivery devices for therapeutic agents, in which the release of the agents is controlled by a nanoporous membrane.

Fabrication of one such membrane of the disclosure is described in U.S. Pat. No. 9,814,867 and control of the internal diameter of the nanopores of such a membrane is described in U.S. Pat. No. 9,770,412, both patents are hereby incorporated herein by reference.

### Release Rate Control

Devices of the disclosure have the capability to release therapeutic agents, contained within the reservoir, through the nanopores of the membrane at a controlled rate. In some instances, the rate of release of the therapeutic agent is a non-Fickian release rate, i.e., a release rate such as zero order release, which is not proportional to the concentration gradient driving the release. Examples of non-Fickian release rates through nanoporous membranes have been described in U.S. Pat. No. 9,814,867, which is incorporated herein by reference. In certain instances, the therapeutic agent(s) disclosed herein undergo “sustained-release” or “extended-release,” wherein the device maintains drug release over a sustained period of time at a non-Fickian release rate, such as a zero-order release rate.

The exact mechanism by which the nanopores of the membranes control the release rate is not understood in detail. Hypothetically, interactions between the diffusing molecules of the therapeutic agent and the interior wall of the nanotubes could play a role in this release mechanism.

### Manufacture

Methods of manufacture of devices and formulations are described in U.S. Patent Application Publication Nos. 20210246271 and 20220008345, which are incorporated herein by reference.

### Therapeutic Agents and Formulations

Therapeutic agents suitable for embodiments of the disclosure have been described in U.S. Patent Application Publication Nos. 20210246271 and 20220008345, which are incorporated herein by reference. In some embodiments the therapeutic substance is a peptide or protein. In some embodiments the peptide or protein is an incretin mimetic, such as a GLP-1 agonist. In some embodiments the incretin mimetic is semaglutide.

Semaglutide is a human GLP-1 receptor agonist (or GLP-1 analog). The peptide backbone can be produced by yeast fermentation. The lysine at position 26 of semaglutide is modified to contain a hydrophilic spacer and a C18 fatty di-acid. In addition, semaglutide is modified at position 8 to provide stabilization against degradation by the enzyme dipeptidyl-peptidase 4 (DPP-4). A minor modification is made at position 34 to ensure the attachment of only one fatty di-acid. The molecular formula is C₁₈₇H₂₉₁N₄₅O₅₉ and the molecular weight is 4113.58 g/mol. Semaglutide has the following structure:

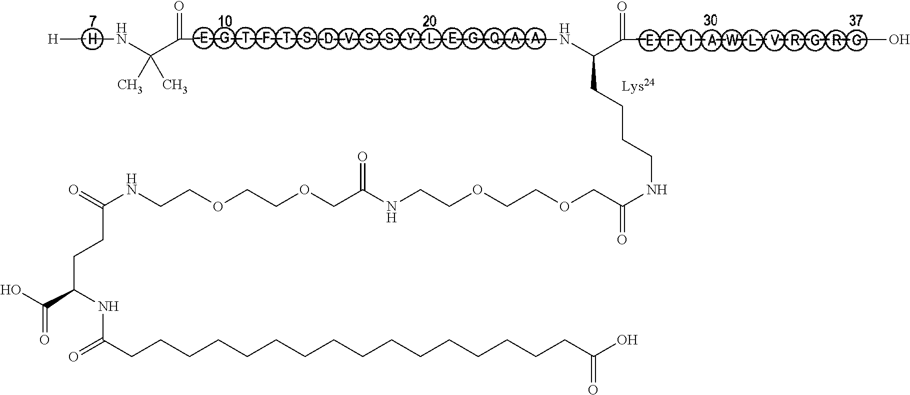

See for example, the European Medicines Agency Dossier dated Dec. 14, 2017, assessment report of OZEMPIC®, incorporated herein by reference. The EMA Assessment Report dossier describes semaglutide as a long-acting analogue of human glucagon like-1 peptide i.e. an Aib8, Arg34-GLP-1 (7-37) analogue substituted on the &-amino group of the lysine residue in position 26 with an(S)-22,40-dicarboxy-10,19,24-trioxo-3,6,12,15-tetraoxa-9,18,23-triazatetracontan-1-oyl side chain. The side chain consists of two 8-amino-3,6-dioxaoctanoic acid (ADO) spacers, one γ-glutamic acid (Glu) spacer, and a fatty diacid (1,18-octadecanedioic acid). Semaglutide is produced using recombinant DNA technology in yeast (Saccharomyces cerevisiae) and chemical modification.

Many peptides and proteins are prone to formation of aggregates when in solution. This propensity tends to increase with increasing concentrations. These aggregates can be permanent or reversible, covalently bonded, or non-covalently bonded, soluble or insoluble, and random or organized, such a beta sheets or fibrils. In many instances these aggregates are undesirable, for instance when they are irreversible and lose pharmaceutical activity, or when they cause detrimental effects, such as excessive increase in viscosity of a formulation.

These aggregations are well-known and well-studied in the literature and are a significant obstacle in the preparation of high concentration formulations of peptides and proteins. Many formulation approaches have been described in the literature, often including excipients such as surfactants.

Semaglutide (CAS #910463-68-2) is an incretin-mimetic, structurally analogous to GLP-1. Semaglutide is known to have a propensity for aggregation, forming fibrils as well as random High Molecular Weight Species. As discussed below, other incretin mimetics are use useful in the present disclosure.

Typically, pharmaceutical preparations of semaglutide do not exceed 1% (w/w) or 10 mg/ml (U.S. Pat. No. 11,318,191, incorporated herein by reference). Surprisingly, however, it has now been found that significantly higher concentrations can be prepared and used in pharmaceutical applications without undue loss to such aggregation phenomena or even degradation. Formulations up to 25% w/w have been prepared and tested for pharmaceutical utility. A measure of stability in this respect that has been cited in the literature is 80% retention of a pure GLP-1 analog (Or loss of no more than 20%) after 3 months of quiescent storage at 25° C. (U.S. Pat. No. 10,888,605). In other words, a formulation of an incretin mimetic (e.g., semaglutide) possess chemical stability if its covalent bonds are intact in at least 80% (w/v) of the incretin mimetic compound after storage for 3 months at 25° C.

Embodiments of this disclosure include formulations with more than 1% semaglutide, such as at least 2%, or at least 5%, or at least 10%, or at least 15%, or at least 20%, or at least 20%, or at least 30% that meet this stability criterion. All percentages are on a w/w basis. In some instances, the concentration of range of semaglutide is 1.1% to about 35% w/w or 12% to about 35% w/w.

In some embodiments the semaglutide is present with a counterion. In some embodiments the counterion is acetate or another pharmaceutically acceptable salt. In some embodiments, the higher concentrations of semaglutide are achieved by adding a basic pH adjusting agent (e.g., NaOH) to the dissolution solvent before adding the semaglutide. Such a dissolution process significantly reduces the viscosity and improves ease of mixing.

A skilled person would appreciate that the therapeutic agent can be a different incretin mimetic other than semaglutide. Incretin mimetics include, for example, liraglutide, semaglutide, dulaglutide, liraglutide, tirzepatide, exenatide, albiglutide, lixisenatide, cotadutide, sitagliptin, saxagliptin, alogliptin, and linagliptin.

In some embodiments, the concentration of the incretin mimetic is at least 1.1%, at least 2%, at least 5%, at least 10%, at least 15%, at least 20%, at least 25%, at least 30% or at least 35% w/w.

In some embodiments the solvent is purified water. In some embodiments a cosolvent is present, such as poly-ethylene glycol, or propylene glycol. In some embodiments the cosolvent is present in up to 50% w/w of the mixed solvent i.e., a mixed aqueous solvent. In certain other aspects, the cosolvent is present from about 1% to about 50%, such as 5%, 10%, 15%, 20%, 25%, 30%, 35%, 40%, 45%, or about 50%. Other solvents include organic solvents such as acetone, ethanol, methanol, 2-propanol, ethyl acetate, isopropyl acetate, methyl ethyl ketone, 1-butanol, and tert-butanol.

In some embodiments no additional pharmaceutical excipients are present in addition to the above mentioned counterions and solvents or cosolvents.

In some embodiments additional pharmaceutical excipients are present.

In some embodiments pH adjusting excipients or components are present, such as acids, bases or buffer systems. In some embodiments the pH is between 6 and 10, such as 6, 7, 8, 9 or 10 and values in-between. In some embodiments the pH is between 7 and 9. In some embodiments the pH is between 7 and 8. In some embodiments the pH adjusting components are non-soluble components such as acidic or basic ion exchange resins. In some embodiments the formulation of the disclosure comprises a buffer, such as phosphate buffer, TRIS, or no buffer. In some embodiments the phosphate buffer is a sodium phosphate buffer, such as disodium hydrogen phosphate.

In some embodiments a tonicity agent is present, such as a salt or other co-solute. In some embodiments the tonicity agent is an inorganic compound, such as, but not limited to, NaCl, KCl or CaCl₂). In some embodiments the tonicity agent is an organic compound, such as, but not limited to, a Na, K, or Ca salt of an organic acid or a non-ionic compound, such as, but not limited to sugars, alcohols, or sugar-alcohols. Non-limiting examples of tonicity agents include dextrose, glycerin, and mannitol. In general, a tonicity agent may be present at about 0.1% to about 10% w/w.

In some embodiments a surfactant is present. The addition of surfactant(s) can significantly increase the rate of release of therapeutic agents into an environment of use from implantable devices of the disclosure. Any type of pharmaceutically acceptable surfactant may be used in embodiments of the disclosure, including, but not limited to, natural and synthetic surfactants, cationic surfactants, anionic surfactants, zwitterionic surfactants and non ionic surfactants.

Non-ionic surfactants include fatty alcohol ethoxylates, octaethylene glycol monododecyl ether, pentaethylene glycol monododecyl ether, alkylphenol ethoxylates, nonoxynols, Triton X-100, fatty acid ethoxylates, ethoxylated amines and/or fatty acid amides, polyethoxylated tallow amine, cocamide monoethanolamine, cocamide diethanolamine, terminally blocked ethoxylates, poloxamers, fatty acid esters of polyhydroxy compounds, fatty acid esters of glycerol, glycerol monostearate, glycerol monolaurate, fatty acid esters of sorbitol, Spans, sorbitan monolaurate, sorbitan monostearate, sorbitan tristearate, Tweens, Tween 20, Tween 40, Tween 60, Tween 80, fatty acid esters of sucrose, alkyl polyglucosides, alkyl polyglycosides, decyl glucoside, lauryl glucoside, octyl glucoside.

Some embodiments of the disclosure include more than 1 surfactant.

The mechanism of release rate acceleration by surfactants is not clear and may or may not be related to the critical micelle concentration (CMC) of the surfactants, or to the level to which they can lower surface tension.

In some embodiments an antioxidant or free radical scavenger is present.

In some embodiments a peptide stabilizing agent is present. In some instances, the stabilizing agent is a surfactant. In some embodiments the stabilizing agent is a complexing agent, such as hydroxypropyl alpha cyclodextrin, hydroxypropyl beta cyclodextrin, hydroxypropyl gamma cyclodextrin, alpha cyclodextrin, beta cyclodextrin, or gamma cyclodextrin. In some embodiments the stabilizing agent is an antioxidant or a free-radical scavenger.

In some embodiments a cyclodextrin is present at about 1% to about 20% w/w such as 1, 2, 3, 4, 5, 6, 7, 8, 9, 10, 11, 12, 13, 14, 15, 16, 17, 18, 19 or 20% w/w or about 1% to about 15%, such as about 10%.

In some embodiments the formulations are contained within reservoirs of devices of the disclosure.

Some embodiments of the disclosure include methods to treat subjects in need of treatment with formulations or devices of the disclosure.

The formulations of the present disclosure are stable with minimal or no loss due to aggregation or degradation of the active ingredient(s) such as semaglutide or other incretin memetic. In certain instances, the formulations having a concentration of semaglutide of greater than 1% w/w up to 35% w/w, which are stable from 1 month up to 2 years, such as up to about 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, and/or 24 months. In some instances, the formulations are stable for more than 2 years.

Advantageously, these stabilities allow long time periods for implantation of the devices of the disclosure. In certain instances, the time periods in which the device are implanted extend up to 1 year or even 2 years and deliver effective and efficacious medical treatments for a variety of diseases.

In some embodiments, the formulations, methods, and devices are used for medical indication and treatments, such as treatment or prophylactic treatment of diabetes, hyperglycemia, type 2 diabetes, glucose intolerance, type 1 diabetes, non-insulin dependent diabetes, gestational diabetes, and/or the reduction of HbA1c. Such indications include delaying or prophylactically treating diabetic disease progression or delaying the progression of glucose intolerance to insulin requiring type 2 diabetes, and/or delaying the progression of non-insulin requiring type 2 diabetes.

“Diabetes” or “diabetes mellitus” refers to the group of metabolic diseases having raised blood sugar levels for an extended period of time. Diabetes includes type 1 diabetes, resulting from a lack of insulin production, and type 2 diabetes, which results from insulin resistance where the cells no longer respond to insulin and can progress to a lack of insulin. Other forms of diabetes are known to one of skill in the art.

The indications also include prevention, prophylactic treatment, or treatment of eating disorders, such as obesity, e.g., by decreasing food intake, reducing body weight, suppressing appetite, inducing satiety; treating, prophylactically treating or preventing binge eating disorder, bulimia nervosa, and/or obesity induced by administration of an antipsychotic or a steroid; reduction of gastric motility; and/or delaying gastric emptying. In certain instances, the indication is as an adjunct to diet and exercise to improve glycemic control in adults with type 2 diabetes mellitus.

The doses suitable for the treatment of diabetes or obesity can provide any suitable mean steady-state plasma concentration of the therapeutic agent in the subject. For example, the mean steady state plasma concentration can be from 10 pg/ml to 10,000 ng/ml. In some embodiments, the mean steady state plasma concentration for semaglutide can be from 10 to 300 ng/ml or 65.0 ng/ml to 123.0 ng/ml, or 111.1 ng/ml to 222.1 ng/ml.

In certain embodiments, the semaglutide concentration is sufficient to achieve an average or minimum circulating blood plasma level of semaglutide of at least 100 ng/ml for of at least about 1 month, at least about 3 months, or at least about 6 months, at least about 8 months, at least about 12 months, at least about 18 months or at least about 24 months or more.

The therapeutic agent can be an incretin mimetic, or GLP-1 analogs or GLP-1 agonists which refer to agents that act like incretin hormones such as glucagon-like peptide-1 (GLP-1). They bind to GLP-1 receptors and stimulate glucose dependent insulin release, therefore acting as antihyperglycemics. Incretin mimetics include, for example, liraglutide, semaglutide, dulaglutide, liraglutide, tirzepatide, exenatide, albiglutide, lixisenatide, cotadutide, sitagliptin, saxagliptin, alogliptin, and linagliptin.

In certain aspects, the incretin mimetic is present at a concentration of about 1%, such as at least 2%, or at least 5%, or at least 10%, or at least 15%, or at least 20%, or at least 20%, or at least 30%.

In some embodiments a peptide stabilizing agent is present. In some instances, the stabilizing agent is a surfactant. In some embodiments the stabilizing agent is a complexing agent, such as hydroxypropyl alpha cyclodextrin, hydroxypropyl beta cyclodextrin, hydroxypropyl gamma cyclodextrin, alpha cyclodextrin, beta cyclodextrin, or gamma cyclodextrin. In some embodiments the stabilizing agent is an antioxidant or a free-radical scavenger.

In some embodiments a cyclodextrin is present at about 1% to about 20% w/w or about 1% to about 15%, such as about 10%.

In one aspect, the therapeutic agent is a dual glucagon-like peptide-1 and glucagon receptor agonist, such as cotadutide and liraglutide.

Provided herein are methods of improving glycemic control, reducing weight, and treating type 2 diabetes mellitus (T2DM) in human patients comprising administering GLP-1/glucagon agonist peptides, such as cotadutide. The chemical formula of cotadutide is C₁₆₇H₂₅₂N₄₂O₅₅, and it has a molecular weight of 3728.09, a CAS number of 1686108-82-6 and has the following sequence, HSQGTFTSDX10SEYLDSERARDFVAWLEAGG-acid, wherein X10=lysine with a palmitoyl group conjugated to the epsilon nitrogen, through a gamma glutamic acid linker (i.e., K (gE-palm)).

The chemical formula of liraglutide is C₁₇₂H₂₆₅N₄₃0₅₁ with a molecular weight of 3751.20 a CAS number of CAS 204656-20-2 and has the following sequence, HAEGTFTSDVSSYLEGQAA-[Lys ([Palm]-[g-Glu]-)]-EFIAWLVRGRG-acid, having an addition of a glutamic acid-spaced palmitic acid to the ε-amino group of lysine in position 26.

In one aspect, the therapeutic agent is LY3437943, which is a novel triple agonist peptide at the glucagon receptor (GCGR), glucose dependent insulinotropic polypeptide receptor (GIPR), and glucagon-like peptide-1 receptor (GLP-1R).

The therapeutic agent can be administered by any means known to one of skill in the art. For example, the therapeutic agent can be administered via the implantable drug delivery system of the present invention. In some embodiments, the present invention provides a method of administering a therapeutic agent to a subject in need thereof, the method including implanting in the subject an implantable drug delivery system of the present invention containing a pharmaceutical composition of the present invention including the therapeutic agent and a polymeric buffering agent comprising a polymer functionalized with a plurality of acid groups or a plurality of base groups, wherein the therapeutic agent elutes from the implantable drug delivery system, thereby administering the therapeutic agent.

### EXAMPLES

The following examples are offered to illustrate, but not to limit the claimed invention.

### Example 1

Release rates and stability of 25% semaglutide formulations with cyclodextrin.

### Preparation of the Formulation.

0.7 ml of a solution containing 10% (w/w) cyclodextrin and 0.6% (w/w) NaCl in Water For Injection (WFI) was prepared in a polypropylene centrifuge tube. About 400 mg of powdered semaglutide was added. The drug was dissolved by prolonged mixing on a vortex mixer. During the mixing process, the formulation foamed extensively, significantly impairing the mixing. In order to facilitate mixing the solution was centrifuged on a small tabletop centrifuge multiple times in the process. This removed the foam and greatly improved the mixing process. Once mixing was complete, a viscous, clear solution was obtained after the final centrifugation.

To bring the solution to pH 7.4 about 91 microliter of IN NaOH was added in multiple steps, with mixing, centrifugation and pH measurement after each step. Some loss of the formulation to the pH probe was experienced. The viscosity of the solution dropped significantly during the pH adjustment process. At the end of the process the volume was adjusted to 1.5 mL, followed by mixing and centrifugation.

The solution was sterile filtered by filtration using a 1 ml syringe and a small Pall syringe filter (PN4602).

### Filling of the Reservoirs

Release rate testing was performed using clear polycarbonate devices instead of titanium to allow for visual inspection for visible aggregates, such as fibrils or randomly precipitated aggregates.

Briefly, polycarbonate capsules with an internal reservoir volume of about 50 microliter were fitted with a silicone septum on one end, and with a titanium screw cap holding a titania nanotube membrane on the other end. In order to fill the formulations into the devices, a vacuum was applied to the reservoirs through the membrane, after which the formulations were injected into the reservoirs by piercing the septum with the needle of a syringe filled with formulation attached to a pump system delivering the formulation.

### Release Rate Testing

In-vitro release rate experiments were performed in 4 mL HPLC vials containing 3 mL of PBS at pH 7.4. The vials were incubated in an incubator at 37° C.

### Microbial Control

0.3% sodium azide was added to all formulations and in vitro release rate buffers as an antimicrobial to enable long term release rate experiments without the need for strict aseptic conditions.

### Example 2

Release rates and stability of 15% w/w semaglutide formulations with cyclodextrin.

Identical processes were followed as in Example 1 but using about 240 mg of semaglutide and 54 microliter of IN NaOH.

### Results

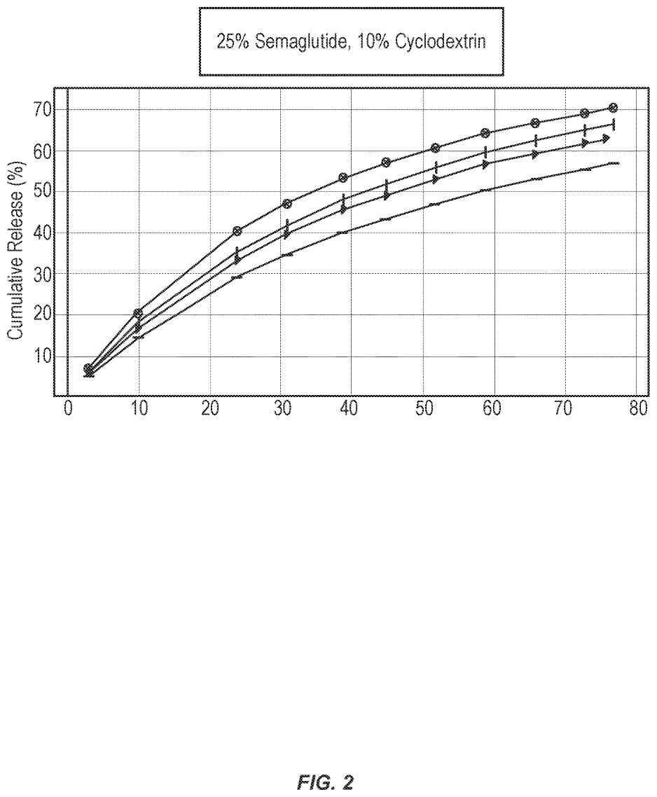

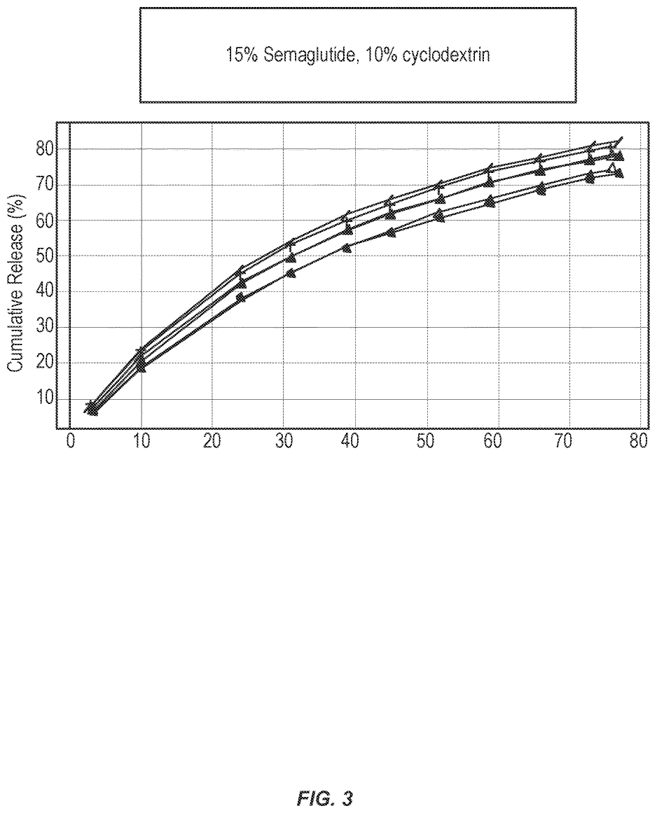

As can be seen in FIGS. 2 and 3, continuous release of the therapeutic agent was observed out to 77 days in examples 1 and 2. FIG. 2 shows 25% w/w semaglutide with 10% w/w cyclodextrin. FIG. 3 shows 15% w/w semaglutide and 10% w/w cyclodextrin. Each trace in the figure represents a separate measurement of a separate vial. At the end of the time period devices were terminated, disassembled and the internal compositions measured.

| | | | Remaining | |
|---|---|---|---|---|
| | | Final | Concentration | % Mass |
| 77 day | | Purity % | (Stdev) | Balance |
| release | Formulation | (Stdev) | mg/mL | (Stdev) |
| | 25% Semaglutide | 86% (+/−0.6%) | 37 (+/−7) | 96% +/− 2.7% |
| | 10% Cyclodextrin | | | |
| | 15% Semaglutide | 87% (+/−1.5%) | 80 (+/−15) | 103% +/− 1.5% |
| | 10% Cyclodextrin | | | |

The starting purity of the semaglutide as received was 94%. Therefore, the total loss in purity over 77 days is in the order of about 7-8%. A loss of purity of 7-8% over 75 days is well within the margin of 20% loss over 3 months.

The mass balance numbers indicate that no appreciable amount of drug was lost in the form of degradants undetectable by HPLC, such a fibrils.

### Example 3

Release rates and stability of 15% w/w semaglutide formulations without cyclodextrin.

### Preparation of the Formulation

0.7 ml of solution containing 0.6% (w/w) NaCl in Water For Injection (WFI) was prepared in a polypropylene centrifuge tube. 45 microliter of IN NaOH was added.

After mixing the ingredients, 240 mg of semaglutide was added. It was found that adding some of the NaOH to the solvent before adding the therapeutic agent significantly reduced the viscosity and improved ease of mixing.

The drug dissolution process was similar to that of Example 1, with the exception of a greatly facilitated mixing process after adding some IN NaOH before adding the therapeutic agent.

Device filling and release rate testing were identical to Example 1.

### Results

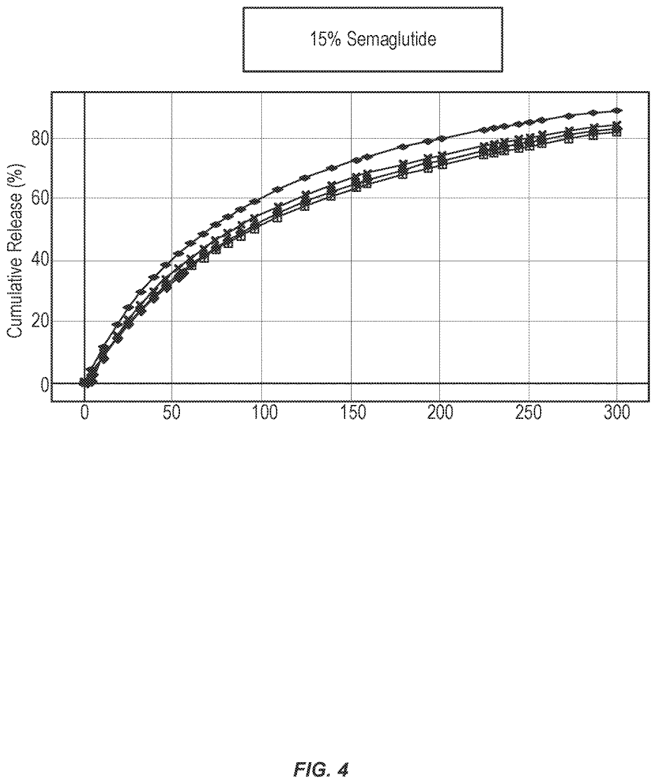

FIG. 4 shows 15% w/w semaglutide in a continuous and ongoing release of the drug out to 300 days. Each trace in the figure represents a separate measurement of a separate vial.

Two devices of the formulation were terminated at 336 days, and the purity and total recovery determined. Purities were 64 and 61%. The mass balance for both devices was 98%, indicating that no appreciable amount of drug was lost in the form of degradants undetectable by HPLC, such a fibrils.

### Example 4

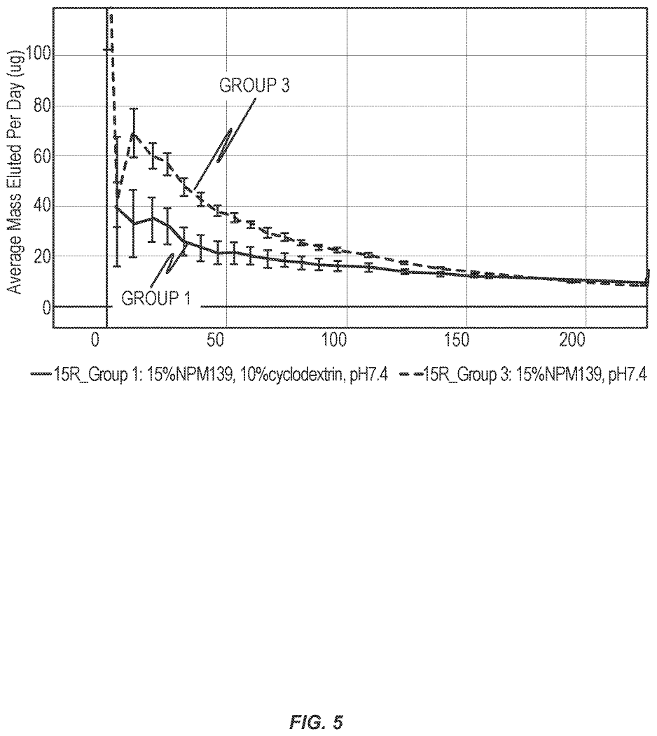

Comparison of release rate profiles of formulations described in Examples 2 and 3 (See, FIG. 5). Group 1, 15% (w/w) semaglutide with 10% (w/w) hydroxypropyl a cyclodextrin is represented in the lower release rate profile. Group 3, 15% semaglutide without any cyclodextrin, is represented by the higher, more rapidly declining profile.

All publications, patents and patent applications cited in this specification are herein incorporated by reference as if each individual publication, patent or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A liquid pharmaceutical formulation having a solvent, the formulation comprising semaglutide having a concentration, wherein the semaglutide concentration (w/w) is more than about 1%.

2. The formulation of claim 1, wherein the semaglutide concentration (w/w) is one of at least 1.1%, at least 2%, at least 5%, at least 10%, at least 15%, at least 20%, at least 25%, at least 30% or at least 35%.

3. The formulation of any one of claims 1-2, wherein the pH of the formulation is between about 6 and about 10.

4. The formulation of any one of claims 1-3, wherein substantially no additional pharmaceutical excipients are present.

5. The formulation of any one of claims 1-3, wherein a tonicity agent is present in the formulation.

6. The formulation of claim 5, wherein the tonicity agent is other than sodium chloride.

7. The formulation of claim 5, wherein the tonicity agent is potassium chloride.

8. The formulation of any one of claims 1-7, wherein the solvent is water.

9. The formulation of any one of claims 1-7, wherein the solvent is an organic solvent or a water-organic solvent mixture.

10. The formulation of any one of claims 1-3, wherein an additional pharmaceutical excipient is present.

11. The formulation of claim 10, wherein the additional pharmaceutical excipient is one or more selected from the group of an antimicrobial, a surfactant, an antioxidant, a free radical scavenger, and a peptide stabilizing agent.

12. The formulation of claim 11, wherein the peptide stabilizing agent is one or more selected from the group of a cyclodextrin and an amino acid.

13. The formulation of claim 12, wherein the cyclodextrin is hydroxy-propyl alpha cyclodextrin.

14. The formulation of claim 12, wherein the amino acid is histidine.

15. A device for sustained release of semaglutide, the device comprising:
a capsule configured for implantation and having a reservoir;
a nanoporous membrane with a plurality of pores; and
a liquid formulation of semaglutide having a solvent and having a concentration of semaglutide disposed within the reservoir, wherein the semaglutide concentration (w/w) is more than about 1%.

16. The device of claim 15, wherein the semaglutide concentration (w/w) is one of at least 1.1%, at least 2%, at least 5%, at least 10%, at least 15%, at least 20%, at least 25% and at least 30% or about 35% w/w.

17. The device of any one of claims 15-16, wherein the pH is between about 6 and about 10.

18. The device of any one of claims 15-17, wherein substantially no additional pharmaceutical excipients are present.

19. The device of any one of claims 15-17, wherein a tonicity agent is present.

20. The device of claim 19, wherein the tonicity agent is other than sodium chloride.

21. The device of claim 19, wherein the tonicity agent is potassium chloride.

22. The device of any one of claims 15-21, wherein the solvent is water.

23. The device of any one of claims 15-21, wherein the solvent is an organic solvent or a water-organic solvent mixture.

24. The device of any one of claims 15-17, wherein an additional pharmaceutical excipient is present.

25. The device of claim 24, wherein the additional pharmaceutical excipient is one or more selected from the group of an antimicrobial, a surfactant, an antioxidant, a free radical scavenger, and a peptide stabilizing agent.

26. The device of claim 25, wherein the peptide stabilizing agent is one or more selected from the group of a cyclodextrin and an amino acid.

27. The device of claim 26, wherein the cyclodextrin is hydroxy-propyl alpha cyclodextrin.

28. The device of claim 26, wherein the amino acid is histidine.

29. A method for treating a subject with semaglutide in need of treatment, comprising:
providing a device for sustained release of semaglutide,
the device comprising:
a capsule configured for implantation and having a reservoir;
a nanoporous membrane with a plurality of pores; and
a liquid formulation of semaglutide having a solvent and having a concentration of semaglutide disposed within the reservoir, wherein the semaglutide concentration (w/w) is more than 1%.

30. The method of claim 29, wherein the semaglutide concentration (w/w) is one of at least 1.1%, at least 2%, at least 5%, at least 10%, at least 15%, at least 20%, at least 25% and at least 30% or about 35% w/w.

31. The method of any one of claims 29-30, wherein the pH is between about 6 and about 10.

32. The method of any one of claims 29-31, wherein substantially no additional pharmaceutical excipients are present.

33. The method of any one of claims 29-32, wherein a tonicity agent is present.

34. The method of claim 33, wherein the tonicity agent is a tonicity agent other than sodium chloride.

35. The method of claim 33, wherein the tonicity agent is potassium chloride.

36. The method of any one of claims 29-35, wherein the solvent is water.

37. The method of any one of claims 29-35, wherein the solvent is an organic solvent or a water-organic solvent mixture.

38. The method of any of claims 29-31, wherein an additional pharmaceutical excipient is present.

39. The method of claim 38, wherein the additional pharmaceutical excipient is one or more selected from the group of an antimicrobial, a surfactant, an antioxidant, a free radical scavenger, and a peptide stabilizing agent.

40. The method of claim 39, wherein the peptide stabilizing agent is one or more of selected from the group of a cyclodextrin and an amino acid.

41. The method of claim 40, wherein the cyclodextrin is hydroxy-propyl alpha cyclodextrin.

42. The method of claim 40, wherein the amino acid is histidine.

43. A device for sustained release of a therapeutic agent, the device comprising:
a capsule configured for implantation and having a reservoir;
a nanoporous membrane with a plurality of pores; and
a liquid formulation of the therapeutic agent having a solvent; disposed within the reservoir.

44. The device of claim 43, wherein the therapeutic agent is an incretin mimetic selected from liraglutide, semaglutide, dulaglutide, liraglutide, tirzepatide, exenatide, albiglutide, lixisenatide, cotadutide, sitagliptin, saxagliptin, alogliptin, and linagliptin.

45. A method for flattening the release rate profile of semaglutide from an implantable device, the method comprising:
providing the device, the device comprising:
a capsule configured for implantation and having a reservoir;
a nanoporous membrane with a plurality of pores;
a liquid formulation of semaglutide disposed within the reservoir, the formulation comprising:
a solvent;
a concentration of semaglutide of at least 1%,
a cyclodextrin, and
introducing the device in an environment of use.

46. The method of claim 45, wherein the semaglutide concentration (w/w) is one of at least 1.1%, at least 2%, at least 5%, at least 10%, at least 15%, at least 20%, at least 25% and at least 30% or about 35% w/w.

47. The method of claim 45, wherein the cyclodextrin concentration (w/w) is 1% to about 15%.

48. The method of any one of claims 45-47, wherein the cyclodextrin is hydroxypropyl a cyclodextrin.

49. A method for flattening the release rate profile of a GLP-1 analog from an implantable device, the method comprising:
providing the device, the device comprising:
a capsule configured for implantation and having a reservoir;
a nanoporous membrane with a plurality of pores;
a liquid formulation of GLP-1 analog disposed within the reservoir, the formulation comprising:
a solvent;
a concentration of GLP-1 analog of at least 1%,
a cyclodextrin, and introducing the device in an environment of use.
